(19) United States
(12) Patent Application Publication
(10) Pub. No.: US 2009/0018210 A1
(43) Pub. Date: Jan. 15, 2009

(54) A Method for Promoting Fat Degradation

(75) Inventors: Kumiko Iio, Iwata-shi (JP); Yumika Okada, Iwata-shi (JP)

Correspondence Address: McLELAND PATENT LAW OFFICE, P.L.L.C., 11320 RANDOM HILLS ROAD, SUITE 250, FAIRFAX, VA, 22030, US

(73) Assignee: YAMAHA HATSUDOKI KABUSHIKI KAISHA, Iwata-shi (JP)

(21) Appl. No.: 12/170,005

(22) Filed: Jul. 9, 2008

(30) Foreign Application Priority Data
Jul. 11, 2007 (JP) 2007-182582

(51) Int. Cl. A61K 31/122; A61P 3/00

(52) U.S. Cl. 514/694

(57) ABSTRACT

A novel, new, and highly effective agent for promoting fat degradation that contains astaxanthin and/or an ester thereof as active ingredient is provided. The agent for promoting fat degradation of the present invention has an effect of reducing fat by lipolysis of fat in either a subject who is in a condition in which fat has already accumulated or a subject who is in a condition in which fat has not yet been accumulated. Accordingly, the agent for promoting fat degradation of the present invention can restore hypertrophied adipocytes to a normal size.

### BACKGROUND OF THE INVENTION

1. Field of the Invention

The present invention relates to a method for promoting fat degradation. More specifically, the present invention relates to a method for promoting fat degradation by administering an agent that contains astaxanthin and/or an ester thereof as an active ingredient.

2. Description of the Related Art

Obesity is caused when caloric expenditure is lower than caloric intake and the energy source that thus has not been expended over-accumulates as body fat. The causes of body fat accumulation due to excess energy include lack of exercise, improper eating habits, stress, lipid metabolism abnormality (disorder), excessive secretion of insulin, enlargement of adipocytes, and lack of brown adipocytes. Visceral fat is fat found in the mesentery located within the peritoneal cavity, and visceral adipocytes tend to store fat within the individual cells.

When adipocytes are hypertrophied due to fat accumulation, various types of adipokines (e.g., TNFα, resistin, etc.) are secreted by the adipocytes, causing insulin resistance (i.e., decreased insulin sensitivity). As a result, blood glucose levels can no longer be sufficiently lowered, so that insulin is over-secreted in order to control blood glucose levels, resulting in hyperinsulinemia. When hyperinsulinemia occurs, metabolic syndrome is caused by the action of excess insulin on lipid metabolism and the like.

Adiponectin is one of beneficial adipokines. Adiponectin promotes fatty acid burning and glucose uptake, and alleviates insulin resistance (Takashi Kadowaki et al., “The Role of Adiponectin in Molecular Mechanisms of Diabetes and Cardiovascular Diseases”, proceedings of The 128th Japanese Association of Medical Sciences Symposium on “Diabetes Mellitus and Atherosclerosis”, Dec. 2, 2004, pp. 34-45). The action of adiponectin to burn fatty acids does not take place in adipocytes, but results from activation by adiponectin of AMP-activated protein kinase (AMPK) in the liver and the skeletal muscles In the liver, new production of glucose is suppressed, and fatty acids are burned. In the skeletal muscles, glucose is taken in, and fatty acids are burned.

Differentiation of preadipocytes into adipocytes is accompanied by induction of adiponectin expression. When adipocytes are not hypertrophied, adiponectin is actively secreted from adipocytes. On the other hand, when adipocytes are hypertrophied, the action of adiponectin is decreased by the above-described TNFα and the other adipokines. Furthermore, it has been reported that when adipocytes are hypertrophied, transcription of adiponectin is suppressed, and adiponectin becomes deficient, causing a metabolic abnormality (Takashi Kadowaki et al., ibid).

Carotenoids are naturally-occurring substances having an antioxidative effect, and their various bioactivities have attracted interest. However, few studies have been conducted to investigate the action of carotenoids on obesity and adipocytes. It has been reported only that a carotenoid derived from a vegetable or a fruit suppresses the differentiation induced by insulin of preadipocytes into adipocytes (Japanese Laid-Open Patent Publication No. 2003-95930). However, as described above, when differentiation of preadipocytes into adipocytes is suppressed, adiponectin expression is also suppressed. Thus, it is doubtful whether or not suppression of differentiation into adipocytes relates to an antiobesity effect.

Furthermore, it has been reported that astaxanthin, which is a carotenoid, lowers the concentration of neutral fat in blood (US 2006/293387 A1), suppresses body fat accumulation (US 2007/129436 A1), and reduces body fat (WO 2006/59730).

Also, it has been reported that a naturally-derived substance obtained by fermenting capsicum has an effect of suppressing body fat accumulation or reducing body fat (Japanese Laid-Open Patent Publication No. 2006-265142). Fermented capsicum can reduce body fat by degradation of fat into fatty acids and glycerol. However, there is no prior art which teaches that a carotenoid has the effect of reducing fat by fat degradation, although it is found to have an effect on suppressing differentiation of preadipocytes into adipocytes and an effect of suppressing fat accumulation.

### SUMMARY OF THE INVENTION

It is an object of the present invention to provide a novel agent for promoting fat degradation that can promote lipolysis of fat, thereby suppressing additional accumulation of fat and reducing fat that has already accumulated.

The present invention provides an agent for promoting fat degradation, containing astaxanthin and/or an ester thereof as an active component.

The present invention also provides a method for promoting fat degradation in a mammal subject in need of decrease in fat comprising administering to the subject an amount of astaxanthin and/or an ester thereof effective for promoting fat degradation.

In an embodiment, the fat is subcutaneous fat or visceral fat.

In an embodiment, the mammal subject is a human subject.

In an embodiment, the astaxanthin and/or the ester thereof is derived from a microalga belonging to the genus Haematococcus.

According to the present invention, a novel and highly effective agent for promoting fat degradation is provided. The agent for promoting fat degradation of the present invention effectively promotes lipolysis of fat in adipocytes. When fat degradation is promoted, obesity is reduced, and thus various diseases caused by obesity can be prevented and alleviated. Furthermore, when degradation of fat that has accumulated in adipocytes is promoted, hypertrophied adipocytes can be restored to normal size.

### BRIEF DESCRIPTION OF THE DRAWINGS

FIG. 1 is a graph showing the amount of free glycerol relative to the amount of free glycerol in a control, the free glycerol being generated by fat degradation when a free form or an ester form of astaxanthin is added to human subcutaneous adipocytes.

### DESCRIPTION OF THE PREFERRED EMBODIMENTS

In this specification, the terms “promotion of fat degradation” and “suppression of fat accumulation” refer to clearly different phenomena. “Promotion of fat degradation” refers to reducing the amount of fat by promoting fat degradation. On the other hand, “suppression of fat accumulation” refers to suppressing an increase in the amount of fat, that is, not allowing the amount of fat to increase.

More specifically, “suppression of fat accumulation” means that fat accumulation does not occur in a subject who is in a condition in which fat has already accumulated, and “suppression of fat accumulation” also means that fat accumulation does not occur in a subject who is in a condition in which fat has not yet accumulated. Thus, although improvement in the function of adipocytes cannot be expected, the function can be prevented from further deterioration, and suppression of an increase in body fat can be expected. Generally, ingested fat is transported from the small intestine via the bloodstream into cells of fat tissues and accumulates therein in the form of fatty acids. In this process, suppression of fat accumulation is considered to be, for example, suppressing absorption of the fatty acids into the small intestine and excreting the fatty acids out of the body, or promoting oxidization of the fatty acids, thereby preventing the fatty acids from being taken into the cells as fat.

Conversely, in “promotion of fat degradation”, fat is degraded into fatty acids and glycerol, and thus the amount of fat is reduced in adipocytes in a subject in any condition. Accordingly, for example, hypertrophied adipocytes are diminished in size, and thus the function of the adipocytes is improved. More specifically, abnormality in adipokine secretion is alleviated, which leads to alleviation of lifestyle-related diseases. Furthermore, excessive fat is degraded in non-hypertrophied adipocytes, and thus hypertrophy can be prevented, which leads to prevention of obesity. In either case, an effect of body fat reduction is expected.

In this specification, the terms “fat degradation” is synonymous with “lipolysis”.

The agent for promoting fat degradation of the present invention has an effect of reducing fat by lipolysis of fat in either a subject who is in a condition in which fat has already been accumulated or a subject who is in a condition in which fat has not yet been accumulated. Thus, hypertrophied adipocytes are diminished in size, and thus the function of the adipocytes is improved. More specifically, abnormality in adipokine secretion is alleviated, which leads to alleviation of lifestyle-related diseases. Furthermore, non-hypertrophied adipocytes can be prevented from being hypertrophied, which leads to prevention of obesity. As described above, the agent for promoting fat degradation of the present invention has an effect of reducing fat by lipolysis.

The agent for promoting fat degradation of the present invention contains astaxanthin and/or an ester thereof as an active component. The astaxanthin and/or an ester thereof used in the present invention is a carotenoid represented by the following formula:

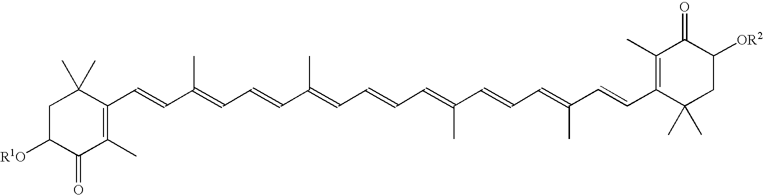

where R¹ and R² are both hydrogen in the case of astaxanthin, and R¹ and R² are each independently a hydrogen atom or a fatty acid residue provided that at least one of R¹ and R² is a fatty acid residue in the case of an ester of astaxanthin. Examples of the fatty acid residue in the ester of astaxanthin include, but are not limited to, saturated fatty acids such as palmitic acid and stearic acid or unsaturated fatty acids such as oleic acid, linoleic acid, α-linolenic acid, γ-linolenic acid, bishomo-γ-linolenic acid, arachidonic acid, eicosapentaenoic acid, and docosahexaenoic acid. The ester of astaxanthin of the present invention can be any mono- or diester, homogeneous or non-homogeneous. Astaxanthin has a structure in which an additional oxo group and an additional hydroxy group are present at each end of a carotene molecule. Conversely, in an ester form (e.g., as obtained in an extract from krill) in which the hydroxy groups at both ends are esterified with an unsaturated fatty acid and the like, the stability of the molecule is better.

Astaxanthin or an ester thereof used in the present invention may be chemically synthesized or derived from a naturally-occurring product. Examples of the naturally-occurring products in the latter case include red yeast; the shell of crustaceans such as Tigriopus (red water flea) and krills; and microalgae such as green algae, which contain astaxanthin and/or an ester thereof. In the present invention, any extract containing astaxanthin and/or an ester thereof produced by any method can be used. Generally, extracts from the aforementioned naturally-occurring products can be used, and the extracts may be crude or purified if necessary. In the present invention, the astaxanthin and/or the ester thereof derived from a microalga belonging to the genus Haematococcus is preferably used. Furthermore, in the present invention, a crude extract, a crushed powder of naturally-occurring products, a purified product or a chemically synthesized product, if necessary, that contains such astaxanthin and/or an ester thereof can be used either alone or in combination. In view of the chemical stability, an ester form of astaxanthin is preferably used.

The route of administration of the agent for promoting fat degradation of the present invention may be either oral or parenteral. The dosage form is selected appropriately according to the route of administration. Examples thereof include parenteral solutions, infusion solutions, powders, granules, tablets, capsules, pills, enteric-coated preparations, troches, liquids for internal use, suspensions, emulsions, syrups, liquids for external use, poultices, nose drops, ear drops, eye drops, inhalants, ointments, lotions, suppositories, and enteral nutrients. These can be used either alone or in combination depending on the condition or the disease to be treated. To prepare these dosage forms, auxiliary substances commonly used in the field of pharmaceutical manufacturing technology, such as excipients, binders, antiseptics, antioxidants, disintegrators, lubricants, and flavoring agents, can be used as necessary.

Examples of a subject in need of decrease in fat include an individual who is obese, an individual who is diagnosed as having metabolic syndrome and obesity, an individual who wants to avoid metabolic syndrome and obesity, and an individual who wants to alleviate the impact of bearing excessive body weight on the condition of the knees.

In this specification, metabolic syndrome is diagnosed according to any of the methods accepted in this field. The diagnostic criteria for metabolic syndrome in Japan are that accumulation of visceral fat is present (waist circumference is 85 cm or more in men or 90 cm or more in women) and that at least two of the following conditions are present: hypertension including borderline hypertension (the maximal blood pressure is 130 mmHg or more or the minimal blood pressure is 85 mmHg or more), hyperlipemia (neutral fat in blood is 150 mg/dl or more) or low HDL (high density lipoprotein) cholesterol (HDL cholesterol is less than 40 mg/dl), and hyperglycemia (fasting blood glucose level is 100 mg/dl or more). Moreover, according to the U.S. guidelines for treatment of hyperlipemia (ATP III: Adult Treatment Panel III, NCEP National Cholesterol Education Program), metabolic syndrome is diagnosed when at least three of the following conditions (i) to (v) are present: (i) waist (abdominal circumference) is 102 cm or more in men or 88 cm or more in women; (ii) neutral fat in blood is 150 mg/dl or more; (iii) HDL cholesterol is less than 40 mg/dl in men or less than 50 mg/dl in women; (iv) blood pressure is such that the maximal blood pressure is 130 mmHg or more or the minimal blood pressure is 85 mmHg or more; and (v) fasting blood glucose level is 110 mg/dl or more. Furthermore, the diagnostic criteria for metabolic syndrome according to the WHO are that hyperinsulinemia is present or fasting blood glucose level is 110 mg/dl or more, plus that at least two of the following conditions (a) to (d) are present: (a) visceral obesity (waist/hip ratio >0.9 (men), >0.85 (women) or a Body Mass Index (BMI) of 30 or more or abdominal circumference of 94 cm or more); (b) lipid metabolism abnormality (neutral fat in blood of 150 mg/dl or more or HDL cholesterol of less than 35 mg/dl in men or less than 39 mg/dl in women); (c) hypertension (140/90 mmHg or more or during treatment with an antihypertensive agent); and (d) microalbuminuria (urinary albumin excretion rate of 20 μg/min or more or urinary albumin/creatinine ratio of 30 mg/g creatinine or more).

The dosage of the agent for promoting fat degradation of the present invention varies depending on the purpose of administration, the individual to whom it is administered (sex, age, body weight, etc.), and the severity and nature of the disease or conditions, and can be determined by a person skilled in the art. Usually, the dosage for an adult in terms of free or unesterified form of astaxanthin may be 0.1 mg to 2 g per day, preferably 4 mg to 500 mg per day in the case of oral administration, while it may be 0.01 mg to 1 g per day, preferably 0.1 mg to 500 mg per day in the case of parenteral administration.

The agent for promoting fat degradation of the present invention can be used not only as pharmaceuticals as described above, but also as products regulated as “quasi-drugs”, cosmetics, food products, nutritional supplements, foods and drinks, and other similar products. When used as quasi-drugs or cosmetics, the agent for promoting fat degradation may be used in conjunction with various auxiliary substances commonly used in the field of quasi-drugs or cosmetics, or other technologies, if necessary. Alternatively, when used as food products, nutritional supplements, or foods and drinks, the agent for promoting fat degradation may be used in conjunction with additives commonly used for food products, for example, sweeteners, spices, seasonings, antiseptics, preservatives, germicides, and antioxidants, if necessary. The agent for promoting fat degradation may be used in a desired form such as solution, suspension, syrup, granule, cream, paste, or jelly, or may be shaped, if necessary. The ratio of the agent for promoting fat degradation contained in these products is not particularly limited, and can be selected appropriately according to the intended purpose, the mode of usage, and the amount of usage.

### EXAMPLES

### Preparation Example 1

### Preparation of Astaxanthin Monoester

An astaxanthin monoester was prepared in the following manner. Haematococcus pluvialis K0084 strain was cultivated at 25° C. under irradiation with light while bubbling a gas containing 3% CO₂ into the culture medium and under nutrient stress condition (i.e. nitrogen source deprivation), and then was encysted. The encysted cells were disrupted by a bead beater, and a lipophilic fraction was extracted with ethanol. The extract contained lipids such as triglyceride, in addition to astaxanthin. The extract was subjected to column chromatography using a synthetic resin absorbent to give a purified product containing an astaxanthin monoester. This purified product was analyzed by HPLC, and it was confirmed that the purified product contained an astaxanthin monoester having a molecular weight of 858 as a main component, but did not contain a free form of astaxanthin, a diester form of astaxanthin, and triglyceride, and that it contained a small amount of diglyceride.

### Example 1

### Examination of the Effect of Astaxanthin on Promotion of Fat Degradation in Human Adipocytes

Human subcutaneous adipocytes (Zen-Bio, Inc.) plated onto a 96-well plate was obtained and pre-cultivated under a 5% CO₂ atmosphere at 37° C. for 5 days in D-MEM/Ham's F12 Medium (1:1, v/v) containing 3% (v/v) fetal bovine serum, 15 mM HEPES (pH 7.4), 33 μM biotin, 17 μM pantothenate, 100 nM human insulin, 1 μM dexamethasone, 100 U/mL penicillin, 100 μg/mL streptomycin and 0.25 μg/mL amphotericin B.

Then, an astaxanthin free form (Wako Pure Chemical Industries, Ltd.: purity 94% or more) and the astaxanthin monoester obtained in Preparation Example 1 were dissolved in dimethyl sulfoxide (DMSO) to prepare stock solutions at a concentration of 3 mM, respectively. The stock solutions were diluted to 30 μM with LIP-2 Assay Buffer (Zen-Bio, Inc.) to prepare test solutions. As a control, 1% (v/v) DMSO solution which was diluted with LIP-2 Assay Buffer was used.

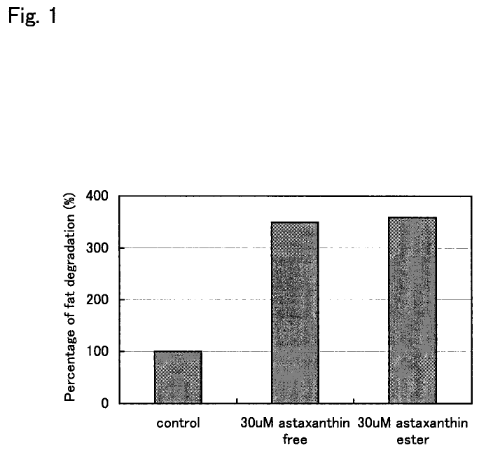

Each of the wells was washed with Wash Buffer (Zen-Bio, Inc.) and the test solutions were added to the wells. These wells were incubated under a 5% CO₂ atmosphere at 37° C. for 3 hours. After the incubation, the amount of free glycerol in the culture supernatant was measured using a Lipolysis Assay Kit (Zen-Bio, Inc.). The percentage of fat degradation promoted is represented as (A/B)×100(%), wherein A is the amount of free glycerol when astaxanthin or a monoester thereof is added, and B is the amount of free glycerol in the control to which astaxanthin or a monoester thereof is not added. The results are shown in FIG. 1.

As can be seen from FIG. 1, the percentage of fat degradation promoted has significantly increased in the adipocytes to which 30 μM astaxanthin or monoester thereof was added, in comparison with the cells (control) to which astaxanthin or a monoester thereof was not added. Accordingly, astaxanthin or a monoester thereof promotes fat degradation in adipocytes.

According to the present invention, a novel and highly effective agent for promoting fat degradation is provided. The agent for promoting fat degradation of the present invention effectively promotes lipolysis of fat in adipocytes of subcutaneous fat and visceral fat. When fat degradation is promoted, obesity is reduced, and thus various diseases caused by obesity can be prevented and alleviated. Examples of such diseases include hyperlipemia, arteriosclerosis, hypertension, myocardial infarction, cerebrovascular disorders, cerebral infarction, angina pectoris, pancreatitis, diabetes, fatty liver, and metabolic disorders. Furthermore, when fat degradation of fat that has accumulated in adipocytes is promoted, hypertrophied adipocytes can be restored to a normal size.

Astaxanthin and/or an ester thereof, which is an active component in the agent for promoting fat degradation of the present invention, has been consumed in food for a long time and has very low toxicity; therefore, astaxanthin and/or an ester thereof has a very high degree of safety. Accordingly, these agents can be used not only as pharmaceuticals, but also as health food products and similar products used prophylactically on a daily basis.

The invention may be embodied in other forms without departing from the spirit or essential characteristics thereof. The embodiments disclosed in this application are to be considered in all respects as illustrative and not limiting. The scope of the invention is indicated by the appended claims rather than by the foregoing description, and all changes which come within the meaning and range of equivalency of the claims are intended to be embraced therein.

What is claimed is:

1. A method for promoting fat degradation in a subject in need of decrease in fat comprising administering to the subject an amount of astaxanthin and/or an ester thereof effective for promoting fat degradation.

2. The method of claim 1, wherein the fat is subcutaneous fat or visceral fat.

3. The method of claim 1, wherein the astaxanthin and/or the ester thereof is derived from a microalga belonging to the genus Haematococcus.
